(19) United States
(12) Patent Application Publication
(10) Pub. No.: US 2007/0004968 A1
(43) Pub. Date: Jan. 4, 2007

(54) Seal for a cannula

(76) Inventors: Frank Bonadio, Bray (IE); Trevor Vaugh, Birr (IE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/290,847

(22) Filed: Dec. 1, 2005

Related U.S. Application Data
(63) Continuation-in-part of application No. 10/902,440, filed on Jul. 30, 2004.
Application No. 10/902,440 is a continuation-in-part of application No. 10/736,234, filed on Dec. 16, 2003.
Application No. 10/902,440 is a continuation-in-part of application No. 10/678,653, filed on Oct. 6, 2003.
Application No. 10/678,653 is a continuation-in-part of application No. 10/133,979, filed on Apr. 29, 2002, now Pat. No. 6,846,287.
Application No. 10/133,979 is a continuation of application No. 09/801,826, filed on Mar. 9, 2001, now abandoned.
Application No. 09/801,826 is a continuation of application No. PCT/IE99/00122, filed on Dec. 1, 1999.
Application No. 10/678,653 is a continuation-in-part of application No. 10/374,523, filed on Feb. 27, 2003.
Application No. 10/374,523 is a continuation of application No. 09/849,341, filed on May 7, 2001, now Pat. No. 6,582,364.
Application No. 09/849,341 is a continuation of application No. 09/688,138, filed on Oct. 16, 2000, now Pat. No. 6,254,534.
Application No. 10/902,440 is a continuation-in-part of application No. 10/665,395, filed on Sep. 22, 2003.
Application No. 10/665,395 is a continuation-in-part of application No. 10/374,523, filed on Feb. 27, 2003.
Application No. 10/374,523 is a continuation of application No. 09/849,341, filed on May 7, 2001, now Pat. No. 6,582,364.
Application No. 09/849,341 is a continuation of application No. 09/688,138, filed on Oct. 16, 2000, now Pat. No. 6,254,534.
Application No. 10/902,440 is a continuation-in-part of application No. 10/374,523, filed on Feb. 27, 2003.
Application No. 10/374,523 is a continuation of application No. 09/849,341, filed on May 7, 2001, now Pat. No. 6,582,364.
Application No. 09/849,341 is a continuation of application No. 09/688,138, filed on Oct. 16, 2000, now Pat. No. 6,254,534.
Application No. 10/902,440 is a continuation-in-part of application No. 10/315,233, filed on Dec. 10, 2002.
Application No. 10/315,233 is a continuation of application No. 09/804,552, filed on Mar. 13, 2001, now Pat. No. 6,578,577.
Application No. 09/804,552 is a continuation of application No. PCT/IE99/00123, filed on Dec. 1, 1999.
Application No. 10/902,440 is a continuation-in-part of application No. 10/133,979, filed on Apr. 29, 2002, now Pat. No. 6,846,287.
Application No. 10/133,979 is a continuation of application No. 09/801,826, filed on Mar. 9, 2001, now abandoned.
Application No. 09/801,826 is a continuation of application No. PCT/IE99/00122, filed on Dec. 1, 1999.
Application No. 09/801,826 is a continuation-in-part of application No. 10/736,234, filed on Dec. 16, 2003.
Application No. 10/902,440 is a continuation-in-part of application No. 10/678,653, filed on Oct. 6, 2003.
Application No. 10/678,653 is a continuation-in-part of application No. 10/133,979, filed on Apr. 29, 2002, now Pat. No. 6,846,287.
Application No. 10/133,979 is a continuation of application No. 09/801,826, filed on Mar. 9, 2001, now abandoned.
Application No. 09/801,826 is a continuation of application No. PCT/IE99/00122, filed on Dec. 1, 1999.
Application No. 10/678,653 is a continuation-in-part of application No. 10/374,523, filed on Feb. 27, 2003.
Application No. 10/374,523 is a continuation of application No. 09/849,341, filed on May 7, 2001, now Pat. No. 6,582,364.
Application No. 09/849,341 is a continuation of application No. 09/688,138, filed on Oct. 16, 2000, now Pat. No. 6,254,534.
Application No. 10/902,440 is a continuation-in-part of application No. 10/665,395, filed on Sep. 22, 2003.
Application No. 10/665,395 is a continuation-in-part of application No. 10/374,523, filed on Feb. 27, 2003.
Application No. 10/374,523 is a continuation of application No. 09/849,341, filed on May 7, 2001, now Pat. No. 6,582,364.
Application No. 09/849,341 is a continuation of application No. 09/688,138, filed on Oct. 16, 2000, now Pat. No. 6,254,534.
Application No. 10/902,440 is a continuation-in-part of application No. 10/315,233, filed on Dec. 10, 2002.
Application No. 10/315,233 is a continuation of application No. 09/804,552, filed on Mar. 13, 2001, now Pat. No. 6,578,577.
Application No. 09/804,552 is a continuation of application No. PCT/IE99/00123, filed on Dec. 1, 1999.
Application No. 10/678,653 is a continuation-in-part of application No. 10/133,979, filed on Apr. 29, 2002, now Pat. No. 6,846,287.
Application No. 10/133,979 is a continuation of application No. 09/801,826, filed on Mar. 9, 2001, now abandoned.
Application No. 09/801,826 is a continuation of application No. PCT/IE99/00122, filed on Dec. 1, 1999.
(60) Provisional application No. 60/433,603, filed on Dec. 16, 2002.
(60) Provisional application No. 60/453,200, filed on Mar. 11, 2003.
(60) Provisional application No. 60/415,780, filed on Oct. 4, 2002.
(60) Provisional application No. 60/428,215, filed on Nov. 22, 2002.
(60) Provisional application No. 60/490,909, filed on Jul. 30, 2003.
(60) Provisional application No. 60/490,909, filed on Jul. 30, 2003.
(60) Provisional application No. 60/490,909, filed on Jul. 30, 2003.
(60) Provisional application No. 60/433,603, filed on Dec. 16, 2002.
(60) Provisional application No. 60/453,200, filed on Mar. 11, 2003.
(60) Provisional application No. 60/631,985, filed on Dec. 1, 2004.
(60) Provisional application No. 60/415,780, filed on Oct. 4, 2002.
(60) Provisional application No. 60/428,215, filed on Nov. 22, 2002.
(60) Provisional application No. 60/490,909, filed on Jul. 30, 2003.
(60) Provisional application No. 60/490,909, filed on Jul. 30, 2003.
(60) Provisional application No. 60/631,985, filed on Dec. 1, 2004.

(30) Foreign Application Priority Data
Feb. 15, 1999 (IE) 990111
Dec. 1, 1998 (IE) 980997
Dec. 16, 1999 (IE) 991053
Oct. 14, 1999 (IE) 990861
May 24, 1999 (IE) 990416
Feb. 15, 1999 (IE) 990112
Feb. 15, 1999 (IE) 990110
Feb. 15, 1999 (IE) 990108
Feb. 15, 1999 (IE) 990107
Dec. 1, 1998 (IE) 980999
Sep. 19, 2002 (IE) 2002/0754
Feb. 18, 2000 (EP) 00650010

(51) Int. Cl. A61B 1/32

(52) U.S. Cl. 600/208

(57) ABSTRACT

A cannula (1) comprises a distal hollow tubular section (2), a proximal instrument insertion section (3), a lip seal (4) for sealingly engaging with an instrument shaft (5), and a duck-bill seal (10) also for sealingly engaging with the instrument shaft (5). The lip seal (4) is movably coupled to the proximal section (3) by a coupling section, provided in the form of a flexible tubular sheath section (6). The distal section (2) defines an access channel (7) for extension of the instrument shaft (5) therethrough. The proximal section (3) has a proximal opening (8) through which the instrument shaft (5) may be inserted into the proximal section (3). The distal end of the flexible section (6) is fixedly attached to the proximal section (3), and the proximal end of the flexible section (6) is fixedly attached to the lip seal (4). The flexible section (6) facilitates relative movement between the lip seal (4) and the proximal section (3) to accommodate lateral movement of the instrument (5) while maintaining the seal between the lip seal (4) and the instrument (5).

This invention relates to a medical device known as a cannula.

The use of a conventional cannula to gain access as a means to see inside the abdomen or to introduce surgical instruments into the abdomen has existed since the late 19^{th} century. A conventional cannula is a rigid tube, which is inserted through the abdominal wall and is held in place by the tension of the abdominal wall itself around the inserted cannula.

Minimally invasive surgery is an evolving surgical method that attempts to reduce the size of incisions required, in many cases dramatically. By using a conventional cannula, the surgeon can gain access with instruments into the abdominal cavity to carry out an operation through a series of very small holes in the abdominal wall. Unlike in the case of “open surgery”, primary retraction is accomplished by lifting the abdominal wall away from the abdominal viscera. This is most often accomplished with the use of gas in a technique known as insufflation.

However there are a number of problems associated with such a conventional cannula. In particular the conventional cannula suffers from the problem of loss of insufflation gas from within the abdomen upon lateral movement of an instrument passed through the cannula.

This invention is aimed at providing an improved cannula which overcomes at least some of the problems associated with a conventional cannula.

### STATEMENTS OF INVENTION

According to the invention there is provided a cannula comprising:
a proximal instrument insertion portion;
a distal tubular portion defining an access channel for extension of an instrument therethrough; and
a first seal for sealingly engaging with the instrument shaft;
the first seal being movably coupled to the proximal portion to facilitate relative movement between the first seal and the proximal portion to accommodate lateral movement of the instrument passing therethrough while maintaining sealing engagement between the first seal and the instrument shaft.

Because the first seal is movable relative to the proximal portion, this enables a certain degree of lateral movement of the instrument to be achieved while still maintaining the seal. Thus the cannula facilitates a certain degree of movement of the instrument, while any loss of insufflation gases from the abdomen is minimised.

In one embodiment of the invention the cannula comprises a coupling portion to movably couple the first seal to the proximal position. The coupling portion may be at least partially substantially tubular. In one case a longitudinal axis of the coupling portion is substantially parallel to a longitudinal axis of the proximal portion. In one embodiment the coupling portion is relatively flexible. The coupling portion may comprise a sheath. In one case the coupling portion is at least partially of a film material. In another case the coupling portion is at least partially of a gelatinous elastomeric material. The gelatinous elastomeric material may comprise a plasticiser. The plasticiser may be selected from the group consisting of naturally derived oils, synthetic oils and liquid oligomers.

In another embodiment the coupling portion is relatively rigid.

In a further embodiment a proximal end of the coupling portion is fixedly attached to the first seal.

A distal end of the coupling portion may be fixedly attached to the proximal portion.

A distal end of the coupling portion may be slidably coupled to the proximal portion. In one case the proximal portion is at least partially substantially spherically shaped. The distal end of the coupling portion may be coupled to the proximal portion in a ball-and-socket arrangement.

In one case the first seal is located externally of the proximal portion. The first seal may be located proximally of a proximal end of the proximal portion. The proximal portion may comprise a proximal opening through which an instrument may be inserted into the proximal portion, and the first seal may be located proximally of the proximal opening.

In one embodiment the first seal comprises a lip seal.

In a further case the cannula comprises a second seal for sealingly engaging with an instrument shaft. The second seal may be fixed relative to the proximal portion.

In one embodiment the second seal is located distally of the first seal. The second seal may be located within the proximal portion. In one case a second seal is located adjacent a proximal end of the proximal portion. The proximal portion may comprise a proximal opening through which an instrument may be inserted into the proximal portion, and the second seal may be located adjacent the proximal opening.

In a further case the second seal comprises a duck-bill seal.

### BRIEF DESCRIPTION OF THE DRAWING

The invention will be more clearly understood from the following description of some embodiments thereof, given by way of example only, with reference to the accompanying drawings, in which:

FIG. 1 is a perspective view of a cannula according to the invention and an instrument in a first position of use;

FIG. 2 is a perspective view of the cannula and the instrument of FIG. 1 in a second position of use;

FIG. 3 is a partially cross-sectional, side view of the cannula and the instrument of FIG. 1 in the first position of use;

FIG. 4 is a partially cross-sectional, side view of the cannula and the instrument of FIG. 1 in the second position of use; and

FIGS. 5 to 8 are views similar to FIGS. 1 to 4 of another cannula according to the invention and the instrument of FIG. 1.

### DETAILED DESCRIPTION

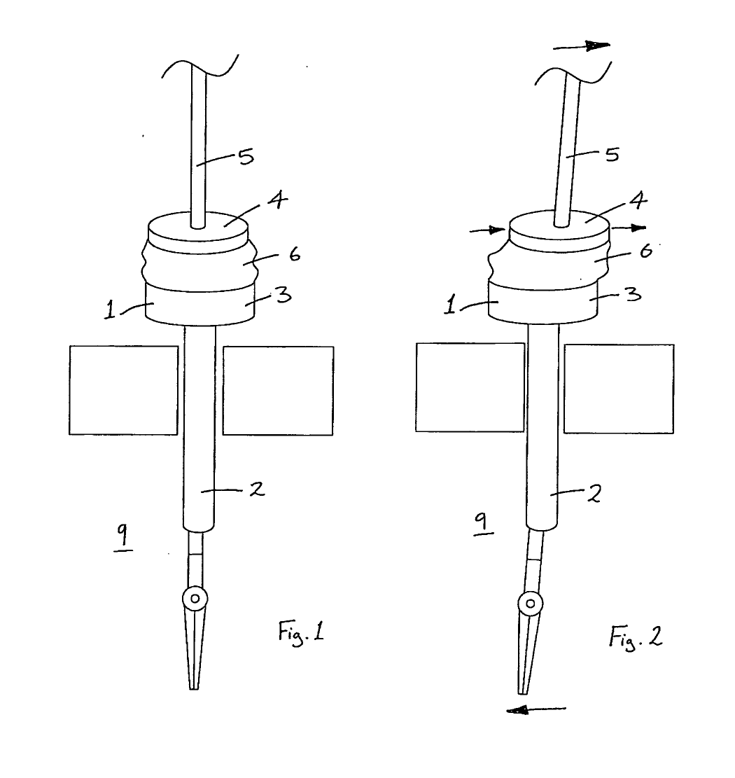

Referring to the drawings and initially to FIGS. 1 to 4 thereof, there is illustrated a cannula 1 according to the invention.

The cannula 1 comprises a distal hollow tubular section 2, a proximal instrument insertion section 3, a proximal seal in this case a lip seal 4 for sealingly engaging with an instrument shaft 5, and a distal seal in this case a duck-bill seal 10 also for sealingly engaging with the instrument shaft 5. The lip seal 4 is movably coupled to the proximal section 3 by a coupling section, which is provided in this case in the form of a flexible tubular sheath section 6.

The distal section 2 defines an access channel 7 for extension of the instrument shaft 5 therethrough.

The proximal section 3 has a proximal opening 8 through which the instrument shaft 5 may be inserted into the proximal section 3.

The lip seal 4 is located externally of the proximal section 3, proximally of the proximal opening 8.

The duck-bill seal 10 is located within the proximal section 3 adjacent the proximal opening 8, and is fixedly attached to the proximal section 3.

The distal end of the flexible section 6 is fixedly attached to the proximal section 3, and the proximal end of the flexible section 6 is fixedly attached to the lip seal 4. The flexible section 6 facilitates relative movement between the lip seal 4 and the proximal section 3 to accommodate lateral movement of the instrument 5 while maintaining the seal between the lip seal 4 and the instrument 5. The cannula 1 thus allows substantial manoeuvrability on insertion of the instrument 5.

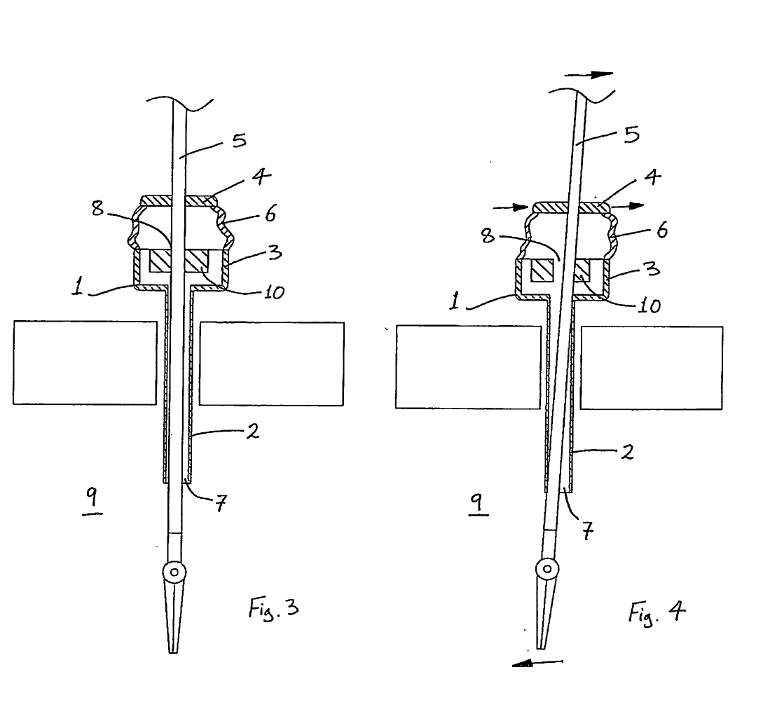

In particular, when the instrument 5 is inserted straight through the cannula I (FIG. 3), the longitudinal axis of the flexible section 6 is parallel to the longitudinal axis of the proximal section 3. If the instrument 5 is tilted to the side, as illustrated in FIG. 4, the flexible section 6 permits lateral movement of the lip seal 4 with the instrument 5. By effectively following the lateral movement of the instrument 5, this ensures that no leakage gap occurs between the instrument 5 and the lip seal 4, and thus the pneumoperitoneum within the abdominal cavity 9 is maintained. In this manner, the cannula 1 provides a solution to the leakage problems encountered by a conventional cannula when an instrument is tilted to the side.

When the instrument 5 is inserted straight through the cannula 1 (FIG. 3), both the lip seal 4 and the duck-bill seal 10 sealingly engage the instrument shaft 5 to prevent loss of insufflation gases from the abdomen 9. When the instrument 5 is tilted to the side (FIG. 4), the duck-bill seal 10 is opened due to the lateral movement of the instrument 5. However the lip seal 4 is free to move laterally with the instrument 5 to maintain the sealing engagement with the instrument shaft 5 to prevent loss of insufflation gases from the abdomen 9.

The flexible section 6 may be formed using any suitable material. For example the flexible section 6 may be at least partially of a gelatinous elastomeric material. An extensive review of gelatinous elastomeric materials is included in U.S. Pat. No. 5,994,450 (Pearce), the entire contents of which are incorporated herein by reference. One such group of gelatinous elastomers may comprise a triblock copolymer A-B-A wherein A is selected from the group consisting of monoalkenylarene polymers and B is a hydrogenated polymer including a plurality of isoprene monomers and a plurality of butadiene monomers. The material may include a plasticiser which may be selected from the group consisting of naturally derived oils, synthetic oils and liquid oligomers.

Alternatively the flexible section 6 may be at least partially of a film material.

The flexible section 6 may be concertinaed to enhance the flexing action.

It will be appreciated that the proximal seal 4 is not limited to being provided in the form of a lip seal. The proximal seal 4 may be provided in any suitable form.

Similarly it will be appreciated that the distal seal 10 is not limited to being provided in the form of a duck-bill seal. The distal seal 10 may be provided in any suitable form.

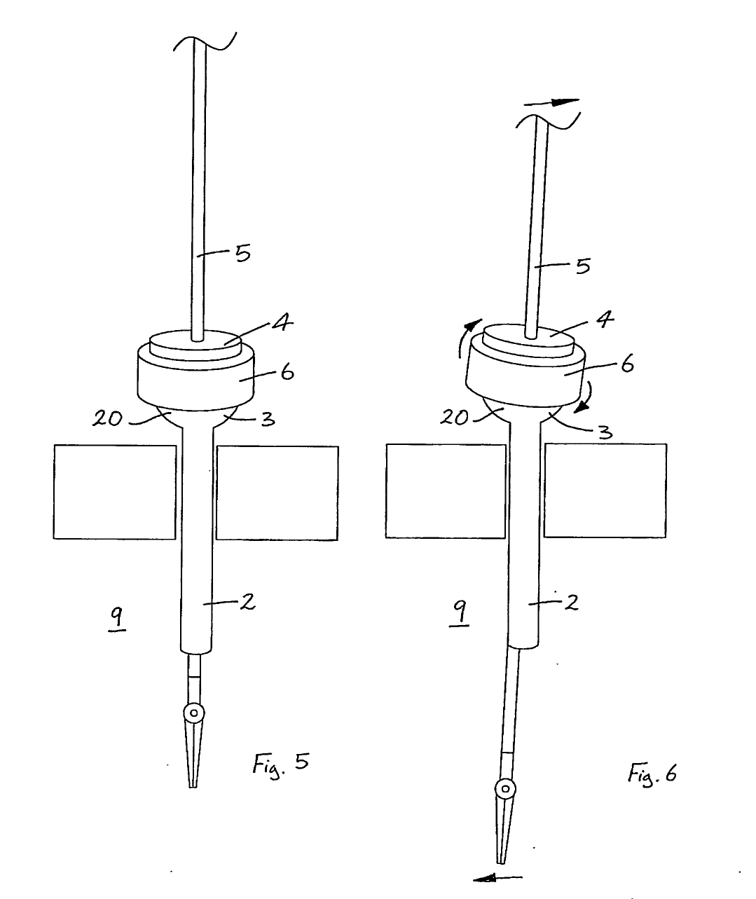

In FIGS. 5 to 8 there is illustrated another cannula 20 according to the invention, which is similar to the cannula 1 of FIGS. 1 to 4, and similar elements in FIGS. 5 to 8 are assigned the same reference numerals.

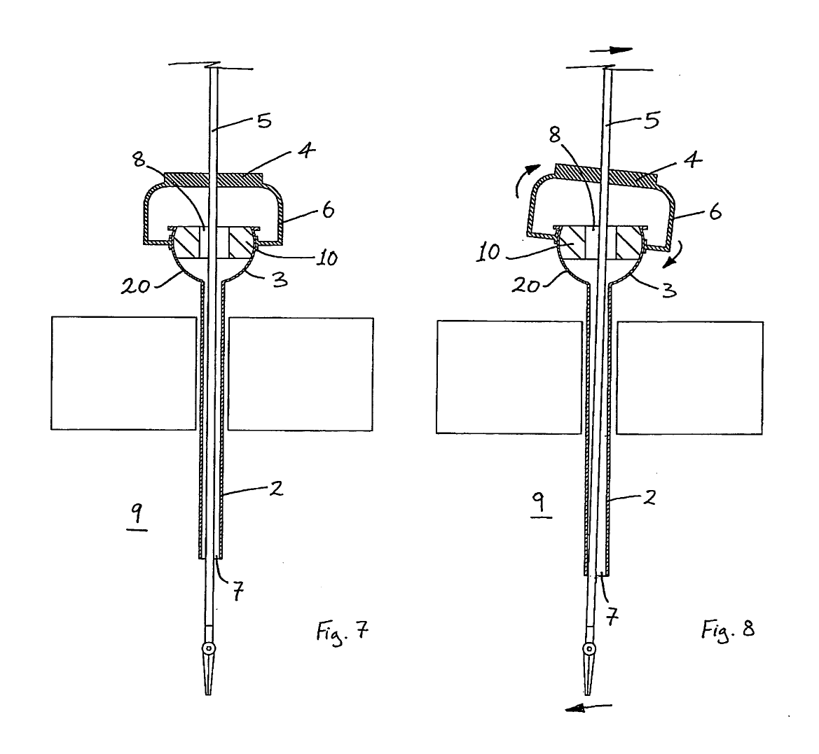

In this case the coupling section is provided in the form of a rigid housing 6, and the proximal section 3 is partially substantially spherically-shaped. The distal end of the housing 6 is slidably coupled the proximal section 3 in a ball-and-socket arrangement, as illustrated in FIGS. 7 and 8. In this manner, the housing 6 facilitates relative movement between the lip seal 4 and the proximal section 3 to accommodate lateral movement of the instrument 5 while maintaining the seal between the lip seal 4 and the instrument 5.

In conventional rigid cannula systems, if the trocar and/or instrument is tilted to one side a leak path is developed through the seal. The cannula of FIGS. 1 to 8 avoid this problem.

The invention is not limited to the embodiments hereinbefore described, with reference to the accompanying drawings, which may be varied in construction and detail.

1. A cannula comprising:
a proximal instrument insertion portion;
a distal tubular portion defining an access channel for extension of an instrument therethrough; and
a first seal for sealingly engaging with the instrument shaft;
the first seal being movably coupled to the proximal portion to facilitate relative movement between the first seal and the proximal portion to accommodate lateral movement of the instrument passing therethrough while maintaining sealing engagement between the first seal and the instrument shaft.

2. A cannula as claimed in claim 1 wherein the cannula comprises a coupling portion to movably couple the first seal to the proximal portion.

3. A cannula as claimed in claim 2 wherein the coupling portion is at least partially substantially tubular.

4. A cannula as claimed in claim 2 wherein a longitudinal axis of the coupling portion is substantially parallel to a longitudinal axis of the proximal portion.

5. A cannula as claimed in claim 2 wherein the coupling portion is relatively flexible.

6. A cannula as claimed in claim 5 wherein the coupling portion comprises a sheath.

7. A cannula as claimed in claim 5 wherein the coupling portion is at least partially of a film material.

8. A cannula as claimed in claim 5 wherein the coupling portion is at least partially of a gelatinous elastomeric material.

9. A cannula as claimed in claim 8 wherein the gelatinous elastomeric material comprises a plasticiser.

10. A cannula as claimed in claim 9 wherein the plasticiser is selected from the group consisting of naturally derived oils, synthetic oils and liquid oligomers.

11. A cannula as claimed in claim 2 wherein the coupling portion is relatively rigid.

12. A cannula as claimed in claim 2 wherein a proximal end of the coupling portion is fixedly attached to the first seal.

13. A cannula as claimed in claim 2 wherein a distal end of the coupling potion is fixedly attached to the proximal portion.

14. A cannula as claimed in claim 2 wherein a distal end of the coupling portion is slidably coupled to the proximal portion.

15. A cannula as claimed in claim 14 wherein the proximal portion is at least partially substantially spherically shaped.

16. A cannula as claimed in claim 15 wherein the distal end of the coupling portion is coupled to the proximal portion in a ball-and-socket arrangement.

17. A cannula as claimed in claim 1 wherein the first seal is located externally of the proximal portion.

18. A cannula as claimed in claim 17 wherein the first seal is located proximally of a proximal end of the proximal portion.

19. A cannula as claimed in claim 18 wherein the proximal portion comprises a proximal opening through which an instrument may be inserted into the proximal portion, and the first seal is located proximally of the proximal opening.

20. A cannula as claimed in claim 1 wherein the first seal comprises a lip seal.

21. A cannula as claimed in claim 1 wherein the cannula comprises a second seal for sealingly engaging with an instrument shaft.

22. A cannula as claimed in claim 21 wherein the second seal is fixed relative to the proximal portion.

23. A cannula as claimed in claim 21 wherein the second seal is located distally of the first seal.

24. A cannula as claimed in claim 21 wherein the second seal is located within the proximal portion.

25. A cannula as claimed in claim 24 wherein the second seal is located adjacent a proximal end of the proximal portion.

26. A cannula as claimed in claim 25 wherein the proximal portion comprises a proximal opening through which an instrument may be inserted into the proximal portion, and the second seal is located adjacent the proximal opening.

27. A cannula as claimed in claim 21 wherein the second seal comprises a duck-bill seal.
